(19) United States
(12) Patent Application Publication
(10) Pub. No.: US 2007/0255201 A1
(43) Pub. Date: Nov. 1, 2007

(54) TIME-INDICATING SYRINGE MIXING DEVICES AND RELATED METHODS FOR STORING AND DISPENSING TWO-PART DENTAL COMPOSITIONS

(75) Inventors: Bruce S. McLean, Sandy, UT (US); Dan E. Fischer, Sandy, UT (US)

Correspondence Address: WORKMAN NYDEGGER, 60 EAST SOUTH TEMPLE, 1000 EAGLE GATE TOWER, SALT LAKE CITY, UT, 84111, US

(73) Assignee: Ultradent Products, Inc., South Jordan, UT (US)

(21) Appl. No.: 11/537,883

(22) Filed: Oct. 2, 2006

Related U.S. Application Data
(63) Continuation-in-part of application No. 11/414,964, filed on May 1, 2006.

(51) Int. Cl. A61M 37/00

(52) U.S. Cl. 604/82

(57) ABSTRACT

Time-indicating syringe mixing systems for mixing, storing, and dispensing a two-part dental composition that becomes less stable upon mixing including a first plunger comprising an elongate stem and a plug at a distal end of the elongate stem, a hollow first chamber configured to contain a first component, a hollow second chamber configured to contain a second component, means for providing a region of increased space between an outside surface of the elongate stem of the first plunger and an interior surface of a wall defining the hollow first chamber, and means for protecting a recorded mixing and/or expiration date from damage or alteration. The inventive syringe mixing systems may be configured as syringe-in-syringe systems and/or syringe-to-syringe systems.

### RELATED APPLICATIONS

The present application is a continuation-in-part of copending U.S. patent application Ser. No. 11/414,964, filed May 1, 2006 and entitled “TIME-INDICATING SYRINGE-IN-SYRINGE MIXING DEVICES AND RELATED METHODS FOR STORING AND DISPENSING TWO-PART DENTAL COMPOSITIONS, the disclosure of which is incorporated by reference in its entirety.

### BACKGROUND OF THE INVENTION

1. The Field of the Invention

The present application is directed to devices for mixing, storing and dispensing time-sensitive dental compositions. More particularly, the application is directed to methods and systems for mixing, storing and dispensing two-part dental compositions that can become destabilized over time within their useful life.

2. The Relevant Technology

Many chemical formulations are packaged in two initially separate parts, often known as A and B components. Separate storage of the A and B components is often necessary where the composition resulting from mixing is unstable over time. For example, a self-etching dental primer composition may be provided in two initially separate parts to prevent the acid component from slowly destabilizing the polymerizable resin component by hydrolyzing off the functional group(s) to which the backbone of the resin is chemically bonded. Although such destabilization may not occur immediately upon mixing, with many such compositions, it is often recommended that the composition be used up or discarded within a certain time period (e.g., 30, 60, or 90 days) after initial mixing.

Because such compositions are typically only used in small amounts in any particular procedure, it can be difficult to determine how much time has elapsed since initial mixing, and whether or not the useful shelf-life of the mixed composition has expired. Simply discarding composition remaining after a single use is effective in preventing use of shelf-life expired product, but is wasteful and unnecessarily expensive. Alternatively, it may be possible to write the date of mixing onto the outside of the mixing or storage syringe. However, such writing can easily become smeared, smudged, altered, worn away, or otherwise rendered illegible, particularly when stored in a typical dental or medical environment. In light of the above, it would be an advantage to provide a time-indicating mixing system for use with a two-part composition that would allow a user to record a mixing date within the system for later reference that would be protected from damage or alteration.

### SUMMARY OF THE INVENTION

The present invention is directed to time-indicating syringe mixing systems for mixing, storing, and dispensing a two-part composition that becomes less stable upon mixing. In one embodiment, the system includes a first plunger comprising an elongate stem and a plug at a distal end of the elongate stem, a hollow first chamber configured to contain a first component, a hollow second chamber configured to contain a second component, means for providing a region of increased space sufficient to receive a label on which is recorded a mixing and/or expiration date (i.e., at least about 0.0005 inch), the region being defined between an outside surface of the elongate stem of the first plunger and an interior surface of a wall that defines the hollow first chamber, and means for protecting the recorded date from damage or alteration. The inventive syringe mixing systems may be configured as a syringe-in-syringe system and/or as a syringe-to-syringe system. The inventive systems advantageously allow a user to mix a two-part composition and to visibly record a mixing and/or expiration date (e.g., on the elongate stem and/or on a label that is inserted into the defined region of increased space) such that the date is carried with the system for later reference. The recorded date is advantageously protected from being damaged or altered. The visible recorded date indicates to the user how much shelf life remains until the mixed composition should be used up or discarded.

One example of a syringe-in-syringe system includes a first plunger, a hollow second plunger configured to contain a first component, and a syringe barrel configured to contain a second component. When assembled, the first plunger is slidably disposed within the hollow second plunger, and the hollow second plunger is slidably disposed within the syringe barrel. The system further includes means for indicating a recorded date when a first component was mixed with a second component and means for protecting the recorded date from damage or alteration.

An example of means for indicating a recorded date comprises an indented substantially flat label surface formed into a portion of the first plunger on which can be recorded a mixing date. The date may be written directly onto the label surface. Alternatively, the date may be written on an adhesive label which is afterwards adhered to the indented substantially flat label surface. An example of means for protecting the recorded date comprises forming at least a portion of the hollow second plunger so as to be transparent or translucent. Pressing the first plunger (including the label surface) into the hollow second plunger protects the recorded date, as the recorded date is covered and protected from damage or alteration by the hollow second plunger. The recorded date advantageously remains visible to the user through the transparent or translucent hollow second plunger.

The inventive syringe-in-syringe mixing system allows a user to mix a two-part dental composition while at the same time providing a record of the mixing date when the first and second components were mixed together. Advantageously, the recorded date is protected from damage or alteration. The system is particularly useful with two-part compositions that expire, lose potency or otherwise become unusable after a certain period of time has elapsed (e.g., a two-part self etching primer having a shelf-life of about thirty days after initial mixing).

Advantageously, the means for protecting the recorded date from damage or alteration may further include a locking mechanism to prevent the first plunger from being withdrawn once inserted into the hollow second plunger, so as to provide further protection against damage or alteration of the recorded date. For this purpose, the first plunger may advantageously comprise a plurality of longitudinally extending interlock ribs or an annular interlock ring formed near its proximal end. The interlock ribs or annular interlock ring are configured for insertion into the hollow second plunger, which includes a corresponding surface that engages the interlock ribs or ring so as to prevent the first plunger from being withdrawn once the ribs or ring have been inserted into the hollow second plunger, thereby locking the first plunger within the hollow second plunger.

The hollow second plunger advantageously includes a transparent or translucent window portion (or the whole hollow second plunger may be transparent or translucent), so as to allow a user to read the recorded date through the hollow second plunger. Because the first plunger is covered by the hollow second plunger, the recorded date is advantageously protected from damage or alteration that might otherwise occur if the recorded date were exposed (e.g., simply written on an outside surface of an exposed syringe barrel). Providing a locking mechanism for preventing withdrawal of the first plunger after insertion advantageously provides additional protection to the recorded date.

These and other advantages and features of the present invention will become more fully apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a perspective view of an exemplary syringe-in-syringe mixing system according to the present invention;

FIG. 2A is a perspective view of an exemplary first plunger of the inventive syringe-in-syringe mixing system including an exemplary locking mechanism;

FIG. 2B is a perspective view of an alternative first plunger of the inventive syringe-in-syringe mixing system including an alternative locking mechanism;

FIG. 3A is a perspective view of an exemplary hollow second plunger of the inventive syringe-in-syringe mixing system including a rupturable membrane at a distal end of the hollow second plunger;

FIG. 3B is a close up cross sectional view of a distal end of the hollow second plunger and rupturable membrane of FIG. 2A;

FIG. 3C is a perspective view of an alternative hollow second plunger of the inventive syringe-in-syringe mixing system including a valve at a distal end of the hollow second plunger;

FIG. 3D is a perspective view of an alternative syringe-to-syringe mixing system;

FIG. 3E is a perspective view of the alternative first plunger included in the syringe-to-syringe mixing system of FIG. 3D;

FIG. 3F is a perspective view of another alternative first plunger for use with a syringe-to-syringe and/or a syringe-in-syringe mixing system;

FIG. 3G is a perspective view of another alternative first plunger for use with a syringe-to-syringe and/or a syringe-in-syringe mixing system;

FIG. 3H is a perspective view of a first plunger and an alternative first syringe barrel for use with a syringe-to-syringe and/or a syringe-in-syringe mixing system;

FIG. 4A illustrates the syringe-in-syringe mixing system of FIG. 1 coupled to a second syringe barrel and associated plunger so as to form a syringe-to-syringe mixing system;

FIG. 4B illustrates a dental practitioner recording the mixing date onto the flat label surface of the first plunger of the mixing system of FIG. 4A prior to mixing the first and second components together;

FIG. 4C illustrates the first plunger being pressed into the hollow second plunger so as to cause the rupturable membrane of the hollow second plunger to break and the first component to mix with the second component;

FIG. 4D illustrates the first plunger in a locked configuration relative to the hollow second plunger;

FIGS. 5A-5B illustrates the exemplary mixing system of FIG. 4A with the user alternatingly pressing the plungers located at the distal and proximal ends of the system so as to cycle the commingled first and second components back and forth so as to form a homogeneous mixture;

FIG. 6 illustrates the user dispensing a portion of the mixed two-part composition onto a pad for application to a desired surface;

FIG. 7A illustrates an alternative syringe-to-syringe mixing system;

FIG. 7B illustrates a dental practitioner recording a mixing and/or expiration date onto a label for insertion into a space defined between the first plunger and the syringe barrel of the syringe-to-syringe mixing system of FIG. 7A; and

FIG. 7C illustrates the first plunger being pressed into the first syringe barrel so as to cause the first component to mix with the second component.

### DETAILED DESCRIPTION OF THE PREFERRED EMBODIMENTS

### I. Introduction

The present invention is directed to time-indicating syringe mixing systems for mixing, storing, and dispensing a two-part composition that becomes less stable upon mixing. The system includes a first plunger comprising an elongate stem and a plug at a distal end of the elongate stem, a hollow first chamber configured to contain a first component, a hollow second chamber configured to contain a second component, means for providing a region of increased space sufficient to receive a label on which is recorded a mixing and/or expiration date, the region being defined between an outside surface of the elongate stem of the first plunger and an interior surface of a wall that defines the hollow first chamber, and means for protecting the recorded date from damage or alteration. The inventive syringe mixing systems may be configured as a syringe-in-syringe system and/or as a syringe-to-syringe system. The inventive systems advantageously allow a user to mix a two-part composition and to visibly record a mixing and/or expiration date (e.g., on the elongate stem and/or on a label that is inserted into the defined region of increased space) such that the date is carried with the system for later reference. The recorded date is advantageously protected from being damaged or altered. The visible recorded date indicates to the user how much shelf life remains until the mixed composition should be used up or discarded.

One embodiment of the invention is directed to a time-indicating syringe-in-syringe mixing system for mixing, storing and dispensing a two-part composition. The system includes a first plunger, a hollow second plunger configured to contain a first component, a syringe barrel configured to contain a second component, means for indicating a recorded date when a first component was mixed with a second component, and means for protecting the recorded date from damage or alteration. When assembled, the first plunger is slidably disposed within the hollow second plunger, and the hollow second plunger is slidably disposed within the syringe barrel. The inventive mixing, storing and dispensing system advantageously allows a user to mix a two-part composition and to visibly record on the system for later reference the date as to when mixing occurred. The recorded date is advantageously protected from being damaged or altered. The visible recorded date indicates to the user how much shelf life remains until the mixed composition should be used up or discarded.

### II. Exemplary Syringe Mixing Systems

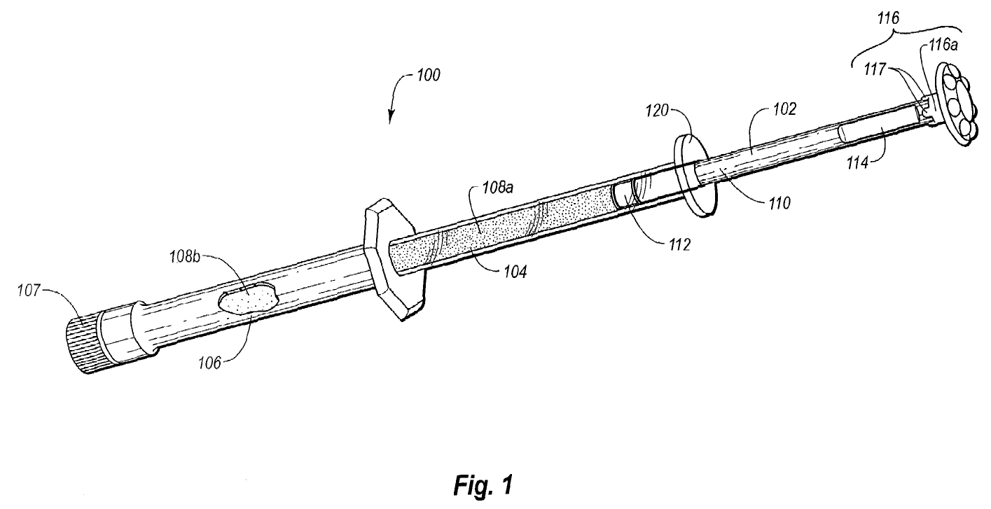

FIG. 1 illustrates an exemplary syringe-in-syringe mixing system 100 according to the present invention. System 100 includes a first plunger 102, a transparent or translucent hollow second plunger 104, and a syringe barrel 106 with a cap 107 at a distal end of syringe barrel 106. First plunger 102 is slidably disposed within hollow second plunger 104, which is slidably disposed within syringe barrel 106. As illustrated, hollow second plunger 104 is configured to contain a first component 108a, and syringe barrel 106 is configured to contain a second component 108b. First plunger 102 includes an elongate stem 110 and a sealing plug 112 at a distal end of stem 110. The sealing plug has a cross-sectional shape corresponding to a cross-sectional shape of the inside of hollow second plunger 104 (e.g., cylindrical). As shown, a substantially flat label surface 114 is formed into cylindrical elongate stem 110 near a proximal end of cylindrical elongate stem 110. The label surface 114 may advantageously be indented or recessed into the elongate stem 110.

First and second components 108a and 108b may each be a liquid, or one may be a solid powder, as dictated by the characteristics of the two-part composition to be mixed. One contemplated two-part composition is a two-part self etching dental primer composition described in U.S. patent application Ser. No. 11/261,171, filed Oct. 28, 2005, and entitled SELF-ETCHING DENTAL PRIMER COMPOSITIONS AND METHODS AND SYSTEMS UTILIZING SUCH COMPOSITIONS, herein incorporated by reference.

Depending on the length of hollow second plunger 104 and first plunger 102 relative to syringe barrel 106, locating label surface 114 near a proximal end of first plunger 102 allows a user to view the label surface 114 and a date recorded thereon even after first plunger 102 and hollow second plunger 104 have be fully inserted into syringe barrel 106 even if syringe barrel 106 is opaque. In other words, forming hollow second plunger 104 and first plunger 102 so as to be approximately equal in length and longer than syringe barrel 106 allows the proximal ends of hollow second plunger 104 and first plunger 102 to extend out of syringe barrel 106, even when fully inserted. For example, if syringe barrel 106 is shorter than hollow second plunger 104 and first plunger 106 by about the length of the label surface 114 (e.g., between about 0.75 inch and about 1 inch), label surface 114 and a date recorded thereon will still be visible to the user when the last of the composition is dispensed. Keeping label surface 114 and a date recorded thereon visible through hollow second plunger 104 is helpful to the user as it indicates whether the shelf life of the composition has expired. Alternatively, or in addition, the barrel 106 may itself be sufficiently transparent or translucent so that the recorded date remains visible even if partially or completely inserted into barrel 106.

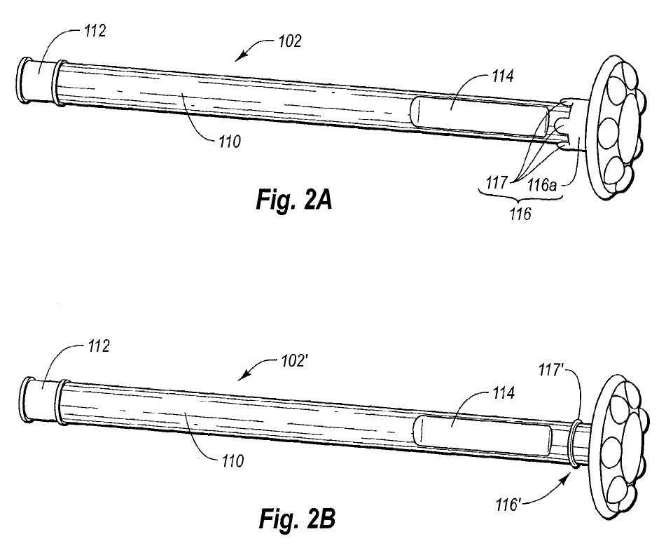

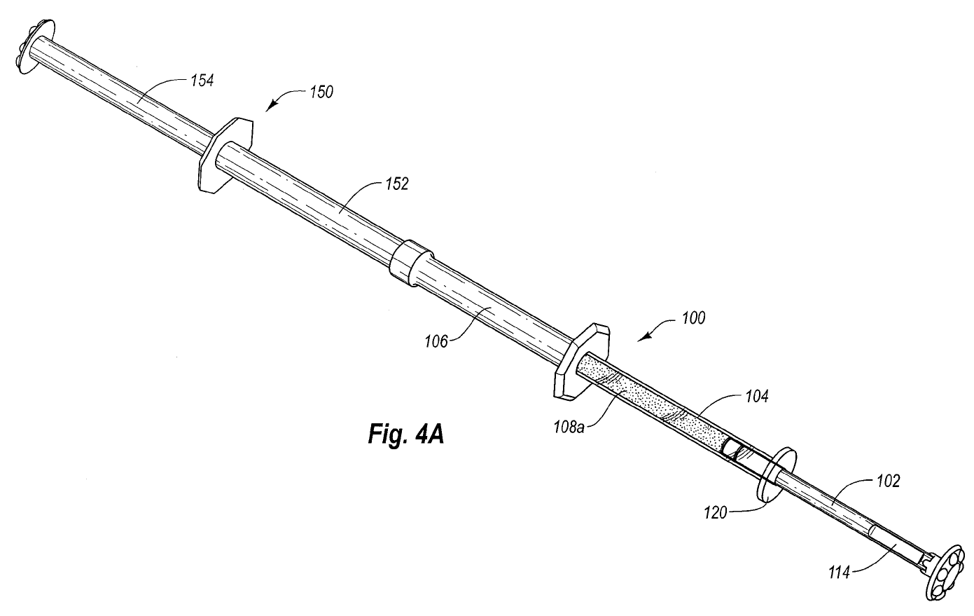

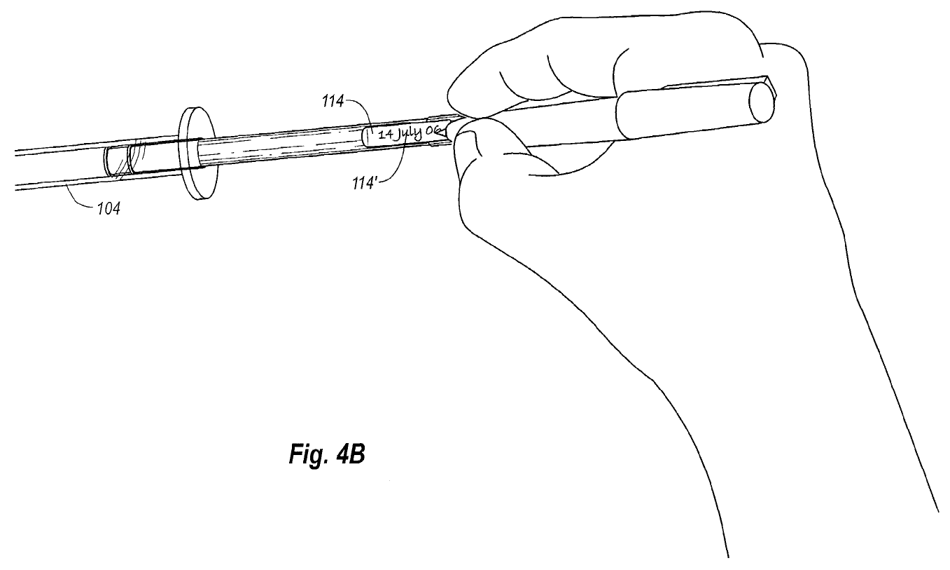

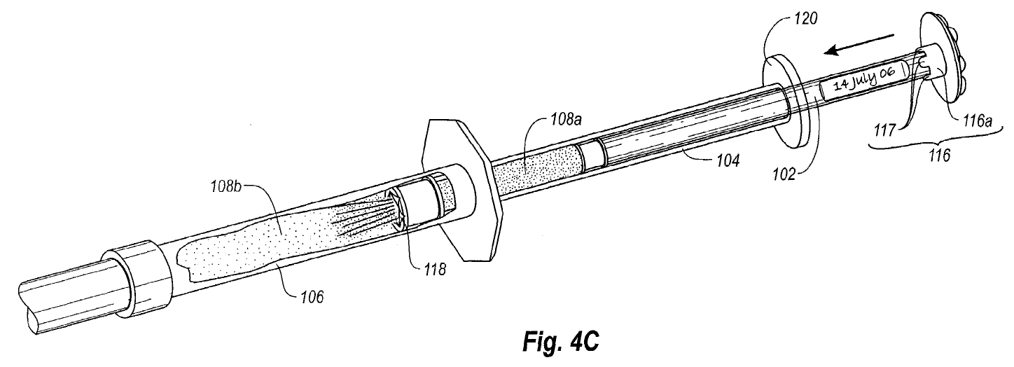

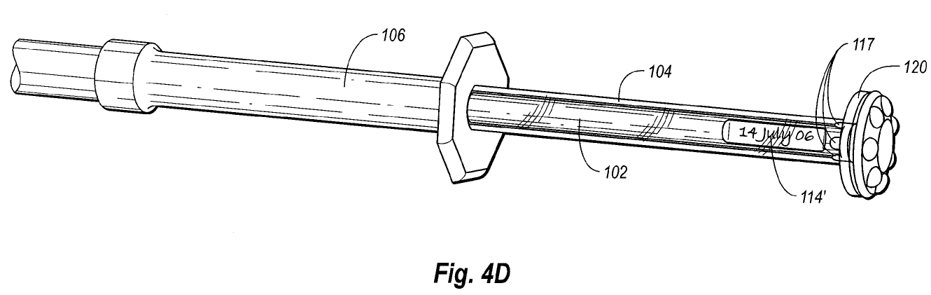

As perhaps best seen in FIG. 2A, a locking mechanism 116 may advantageously be included near a proximal end of first plunger 102 to prevent withdrawal of first plunger 102 from second plunger 104 once inserted. Such a locking mechanism further protects the recorded date from damage or alteration. Illustrated locking mechanism 116 comprises a circumferentially extending portion of enlarged diameter 116a (relative to the remainder of stem 110), with a plurality of longitudinally extending interlock ribs 117. In use, interlock ribs 117 are inserted into hollow second plunger 104, where the ribs 117 bias against the inside wall of hollow second plunger 104. The system is configured such that when first plunger 102 is fully inserted into hollow second plunger 104, circumferentially extending portion 116a rests within flange 120 of hollow second plunger 104, while interlock ribs 117 extend distally into hollow second plunger 104, past flange 120. Because flange 120 provides increased barrel strength relative to the remainder of hollow second plunger 104, little or no deformation occurs to the inside wall of hollow second plunger 104 on account of portion 116a, but deformation is caused by ribs 117, resulting in associated indentations being formed into the inside wall of hollow plunger 104 distal to flange 120, preventing, or at least inhibiting, later removal of first plunger 102 from hollow second plunger 104 (e.g., see FIG. 4D).

FIG. 2B illustrates an alternative first plunger 102′ including a cylindrical elongate stem 110, a sealing plug 112, and an indented substantially flat label surface 114. The principle difference between the first plunger 102′ and first plunger 102 of FIG. 2A and FIG. 1 is that first plunger 102′ includes an alternative locking mechanism 116′ comprising an annular interlock ring 117′ rather than the enlarged diameter portion 116a and plurality of interlock ribs 117 of the embodiment of FIG. 2A. Similar to interlock ribs 117, annular interlock ring 117′ causes the formation of an indentation or groove within the inside wall of hollow second plunger 104. Annular interlock ring 117′ resides in the formed groove, preventing, or at least inhibiting, pull out of first plunger 102 once fully inserted into hollow second plunger 104. Either of locking mechanisms 116 or 116′ further protects information (i.e., the mixing date) recorded on flat label surface 114 from altering by preventing pull out of first plunger 102.

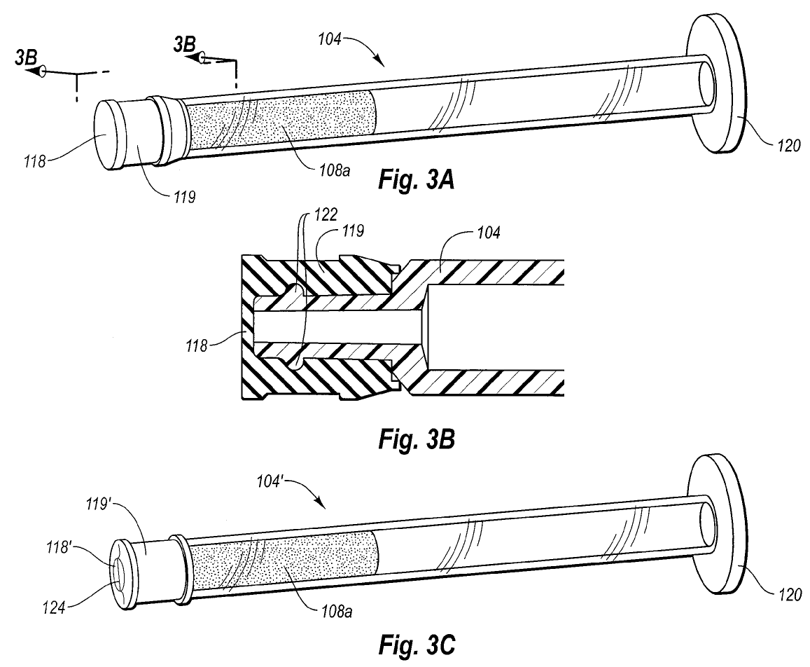

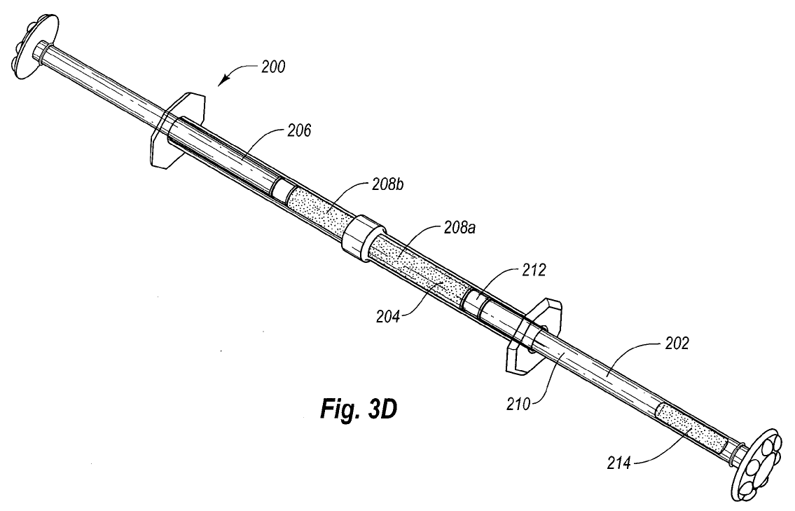

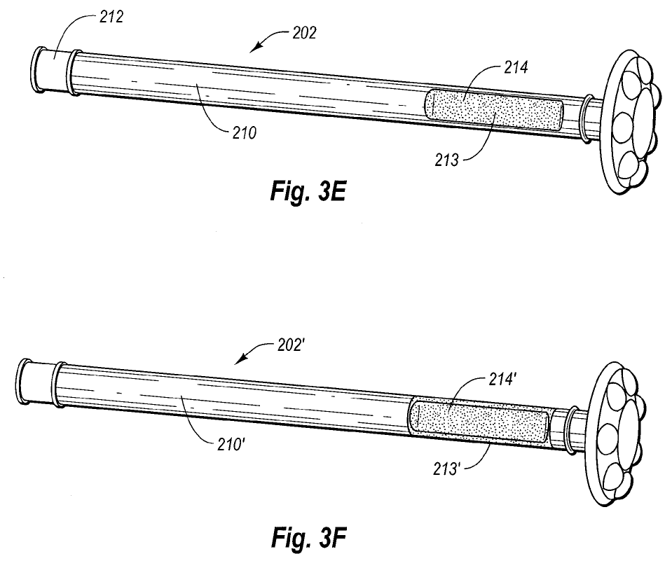

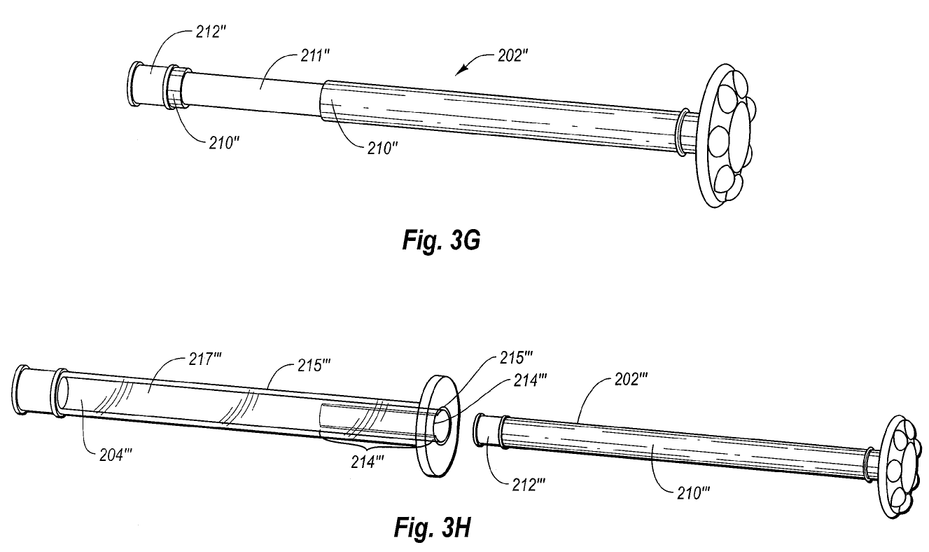

FIG. 3A is a perspective view of hollow second plunger 104 which includes a second sealing plug 119 having a rupturable membrane 118 at a distal end of hollow second plunger 104. Rupturable membrane 118 seals off the distal end of hollow second plunger 104, separating first component 108a from a second component 108b contained within the syringe barrel 106 (see FIG. 1) until the user intentionally ruptures membrane 118, causing first component 108a to be forced into syringe barrel 106, where the two components are mixed together. As seen in FIG. 3B, rupturable membrane 118 initially seals off a distal end of hollow second syringe 104. The distal portion of hollow second plunger 104 over which sealing plug 119 is fitted advantageously includes an enlarged annular ridge 122 that prevents plug 119 from being separated from hollow second plunger 104 during rupture of rupturable membrane 118.

Sealing plug 119 and rupturable membrane 118 may advantageously be formed of a thermoplastic elastomer (TPE), which advantageously provides an excellent seal against syringe barrel 106, while also providing a desired strength to rupturable membrane 118. Advantageously, the system may be configured so that the force required to rupture membrane 118 is approximately equal to the force required to insert and engage the locking mechanism (e.g., interlock ribs 117 or annular interlock ring 117′) of first plunger 102 into hollow second plunger 104. Such a configuration provides a smooth and continuous movement and feel during use of the system as first plunger 102 is pressed into hollow second plunger 104, rupturing membrane 118 and locking first plunger 102 into hollow second plunger 104. Rupturable membrane 118 preferably has a thickness ranging from about 0.0005 inch to about 0.04 inch, more preferably from about 0.002 inch to about 0.025 inch, and most preferably from about 0.005 inch to about 0.015 inch. Of course, the actual thickness of rupturable membrane 118 will depend on the strength and other physical properties of the selected material, along with the configuration and desired level of force required to break the membrane 118 and/or engage the selected locking mechanism. One particularly suitable material from which to form sealing plug 119 and rupturable membrane 118 is ENGAGE, a TPE sold by DuPont-Dow Elastomers located in Wilmington, Del.

FIG. 3C illustrates an alternative hollow second plunger 104′ including an alternative second sealing plug 119′. The principle difference between hollow second plunger 104′ of FIG. 3C and hollow second plunger 104 of FIG. 3B is that plug 119′ includes a pressure sensitive valve (e.g., a one way duck bill valve) 118′ rather than a rupturable membrane. Pressure sensitive valve 118′ acts to initially separate first component 108a within hollow second plunger 104′ from a second component 108b contained within syringe barrel 106 (see FIG. 1). Although pressure sensitive valve 118′ is illustrated as a duck bill one-way valve, any type of valve capable of initially separating the first and second components may alternatively be used. Pressure sensitive valve 118′ includes a slit 124 at a proximal end of plug 119′ through which the first component may be delivered for mixing with the second component.

FIG. 3D illustrates an alternative syringe-to-syringe mixing system 200 according to the present invention. System 200 includes a first plunger 202, a first syringe barrel 204, and a second syringe barrel 206. First plunger 202 is slidably disposed within hollow first syringe barrel 204. As illustrated, hollow first syringe barrel 204 is configured to contain a first component 208a, while second syringe barrel 206 is configured to contain a second component 208b. First plunger 202 includes an elongate stem 210 and a sealing plug 212 at a distal end of stem 210.

As perhaps best seen in FIG. 3E, an indented substantially flat label surface 214 is formed into cylindrical elongate stem 210 near a proximal end of stem 210. Label surface 214 is advantageously indented into stem 210 (i.e., there is a recessed discontinuity along the outer surface of stem 210), and in addition, label surface 214 is overmolded with a thermoplastic elastomer (TPE) material 213. Any TPE material may be used, an example of which is ENGAGE available from DuPont-Dow Elastomers. Other suitable TPE materials are available from J-VON, and/or SARLINK. It has been found that when writing on a molded plastic label surface, it can be difficult to record a date with a ball-point or similar type pen because the label surface is so smooth that it may not “grip” the writing end of the pen. Overmolding or otherwise applying a TPE material over label surface 214 is advantageously helpful when a ball-point pen or similar writing instrument is used to record a mixing and/or expiration date onto label surface 214. The TPE material acts to grip the writing end of such a pen, which makes it much easier to write the date directly onto the TPE covered label surface 214. Felt-tip pens, markers, or even a pencil may also be used to write on the surface. The presence of the TPE material is advantageous as it facilitates writing the desired date with a greater variety of writing instruments.

Because label surface 214 is indented relative to surrounding portions of stem 210, a region of increased space is defined between the stem 210 and the wall of first syringe barrel 204. The recorded mixing and/or expiration date is advantageously protected from being scraped off, worn off, or otherwise altered when first plunger 202 is pressed into first syringe barrel 204.

FIG. 3F illustrates an alternative first plunger 202′. The principal difference relative to plunger 202 of FIG. 3E is that rather than overmolding the TPE material over the label surface, a flexible tube of TPE material 213′ has been seated within a recessed portion of stem 210′, which extends around the full circumference of elongate stem 210′. Tube 213′ includes an indented flat label surface 214′ on which the mixing and/or expiration date may be recorded. The TPE material of label surface 214′ affords the same benefits relative to writing that were discussed above with TPE overmolded label surface 214.

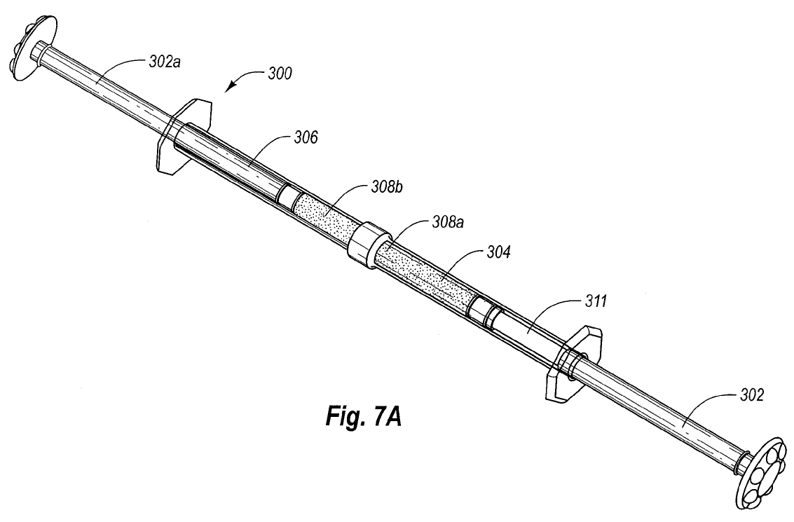

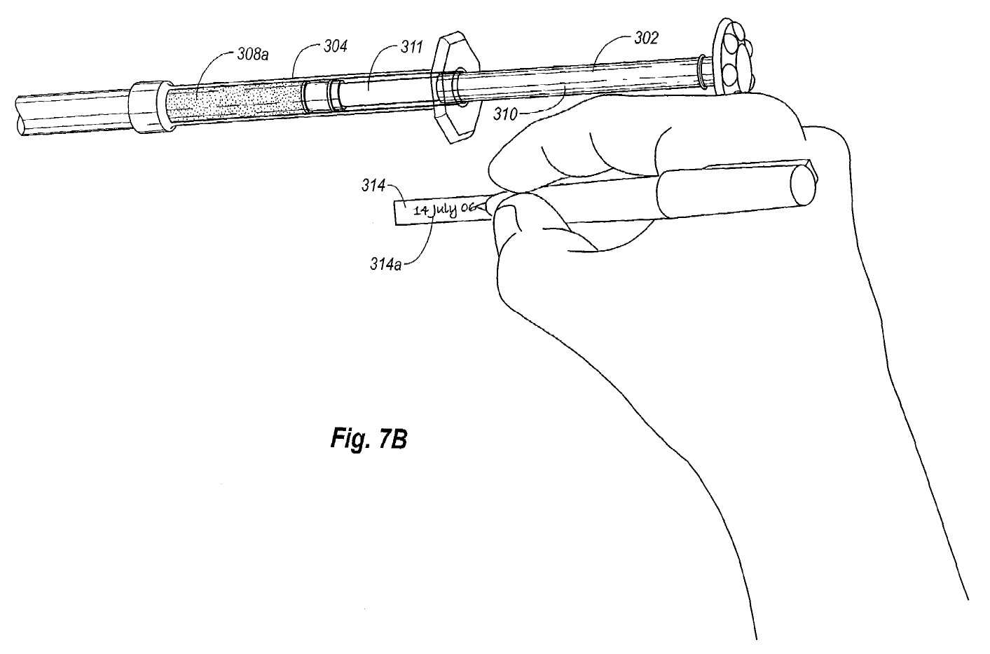

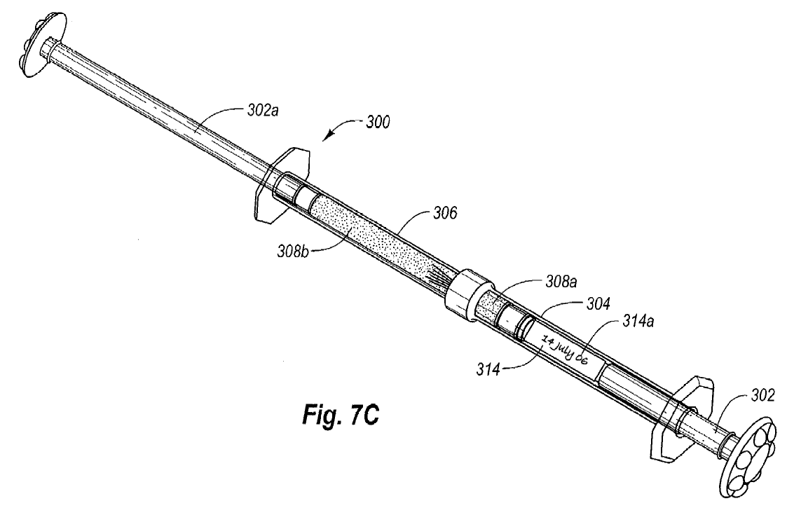

FIG. 3G illustrates another alternative first plunger 202″ including a recessed portion 211″ that includes a cross sectional diameter that is less than the cross-sectional diameter of the remaining adjacent portions of elongate stem 210″. Recessed portion 211″ helps to define a region of increased space between the elongate stem and the interior surface of a wall defining the first syringe barrel when the first plunger 202″ is inserted within the first syringe barrel 204. A label (e.g., a paper label or TPE label with or without an adhesive) on which has been written a mixing and/or expiration date may easily be placed adjacent to recessed portion 211″ prior to pressing first plunger 202″ into a syringe barrel or a hollow second plunger, which acts to trap the label within the defined region of increased space. The label may be adhered to the recessed portion 211″, or alternatively it may include no adhesive so as to simply float free within the defined space, where it is protected from being soiled, damaged or otherwise altered. Such a method is further illustrated in FIGS. 7A-7C, described below.

Recessed portion 211″ is illustrated as being located near the plug 212″, at the distal end of elongate stem 210″, although it may be located anywhere along the length of elongate stem 210″. Locating portion 211″ at the distal end of stem 210″ may be advantageous as it is less likely to be pulled out (and thus potentially exposed) of a syringe barrel or hollow second plunger, which is most helpful in a syringe-to-syringe mixing system. Similarly, the recessed label surfaces of the other embodiments (e.g., FIGS. 1-2B, 3D-3F, and 4A-6) may be located anywhere along the length of the elongate stem of the first plunger. Preferably, recessed portion 211″ is located somewhere along stem 210″ that will be covered (and remain covered) by the first syringe barrel or a hollow second plunger once first plunger 202″ is inserted.

FIG. 3H illustrates another alternative structure comprising means for defining a region of increased space between an outside surface of the elongate stem and the interior surface of the wall of the first hollow chamber (e.g., a hollow second plunger or a first syringe barrel). As shown in FIG. 3H, first plunger 202′″ is configured to slide within a hollow second plunger 204′″. Hollow second plunger 204′″ includes a wall 215′″ defining a hollow chamber 217′″ for containing a first component. A recessed portion 214′″ may be formed within interior surface of wall 215′″ so as to form a recessed portion 214′″ which has an increased diameter relative to an adjacent portion of the hollow chamber 217′″. Although illustrated as comprising a channel or groove within wall 215′″, it will be understood that alternatively the recessed portion 214′″ may extend around the full circumference of the hollow second plunger 204′″, such that no alignment is required between the plunger 202′″ and the hollow second plunger 204′″.

When first plunger 202′″ is inserted within hollow second plunger 204′″, a region of increased space exists between elongate stem 210′″ and wall 215′″ such that a recorded date written on an outer surface of elongate stem 215′″ or written on a label inserted into the region of increased space is protected from damage or alteration once first plunger 202′″ is received within first syringe barrel 204′″. Although it may be possible to use a configuration as illustrated in FIG. 3H with a first syringe barrel in a syringe-to-syringe mixing system, it is preferred that such a configuration be used with a hollow second plunger within a syringe-in-syringe mixing system as the first plunger is typically not withdrawn once inserted into the hollow second plunger, while it may be necessary to at least partially withdraw the first plunger when used within a syringe-to-syringe mixing system, and there is a possibility that a recessed portion (e.g., similar to portion 214′″) may interfere with the ability of the plug 212′″ to seal properly.

First syringe barrel 204 and/or hollow second plunger 204′″ are examples of a first hollow chamber, while second syringe barrel 206 is an example of a second hollow chamber. Although described above principally in the context of a syringe-to-syringe mixing system, one of skill in the art will appreciate that the first plungers described above may alternatively be used in a syringe-in-syringe mixing system, in which the first hollow chamber comprises a hollow second plunger and the second hollow chamber comprises a syringe barrel (e.g., similar to the system illustrated in FIG. 1). Similarly, one of skill in the art will appreciate that any of the first plungers illustrated and described in conjunction with FIGS. 2A-2B may alternatively be used with a syringe-to-syringe mixing system.

### III. Exemplary Method of Use

FIG. 4A illustrates an exemplary syringe-in-syringe mixing system 100 coupled to a second syringe 150 including a second syringe barrel 152 and an associated plunger 154. As seen in FIG. 4B, the user is able to record the mixing date 114′ on label surface 114 prior to fully pressing first plunger 102 into hollow second plunger 104. As illustrated, the user may write directly onto label surface 114 (e.g., with a pen or marker), or alternatively the date may be written on an adhesive label bearing an adhesive (e.g., a pressure sensitive adhesive) which may be subsequently adhered over the label surface 114. In either case, the recorded date 114′ is advantageously not located on a surface of stem 110 that will be rubbed against the inside wall of hollow second plunger 104, which may otherwise cause the date to be smeared, worn away, or otherwise rendered illegible.

Once the mixing date 114′ has been recorded on label surface 114, the user may press first plunger 102 into hollow second plunger 104 so as to compress first component 108a. As shown in FIG. 4C, once a sufficient force is applied, rupturable membrane 118 breaks causing first component 108a to be expressed under pressure from hollow second plunger 104 into syringe barrel 106 where it mixes with second component 108b. As described above, the system may advantageously be configured such that a force required to rupture membrane 118 is approximately equal to a force required to insert and lock locking mechanism 116 (i.e., enlarged diameter portion 116a and interlocking ribs 117) of the cylindrical elongate stem 110 into hollow second plunger 104, although it is not required. FIG. 4D illustrates the system once first plunger 102 has been fully inserted into hollow second plunger 104. In this configuration, first plunger 102 is locked into hollow second plunger 104. As seen, recorded date 114′ is protected by and visible through transparent or translucent hollow second plunger 104.

In the locked configuration as shown, it is difficult, if not impossible, to withdraw first plunger 102 from hollow second plunger 104 without destroying the system. Enlarged diameter portion 116a is disposed within the center of flange 120, while ribs 117 extend distally from flange 120 further into hollow second plunger 104. Because flange 120 has increased barrel strength relative to the area of hollow second plunger 104 immediately distal to flange 120, the inside wall surface of hollow second plunger will be deformed by ribs 117 so as to form a depression into the portion of the inside wall contacted. At the same time, the inside surface of hollow second plunger 104 directly under flange 120 will be deformed only slightly if at all because of the increased barrel strength of the flange region 120 compared to the region contacted by ribs 117. In other words, ribs 117 create an interlock with the inside surface of hollow second plunger 104, preventing, or at least inhibiting, subsequent withdrawal of first plunger 102 from hollow second plunger 104.

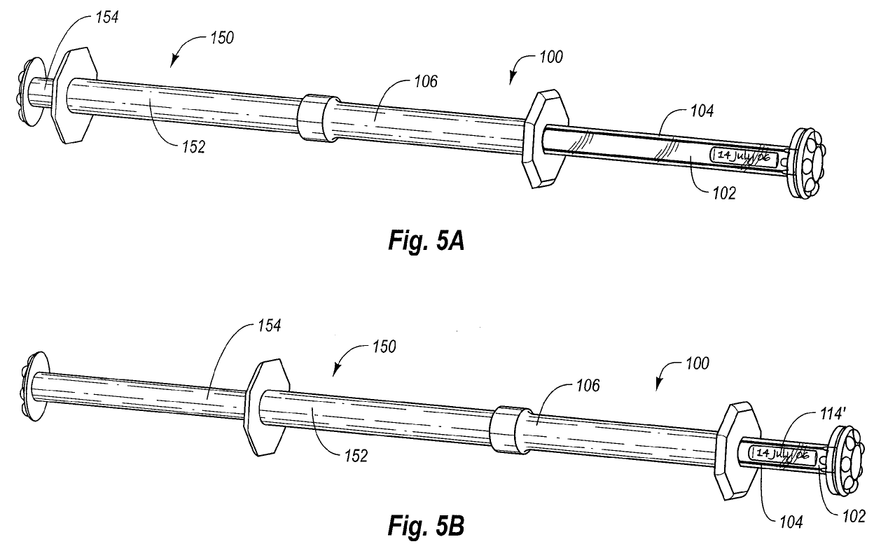

As seen in FIGS. 5A and 5B, the second syringe 150 may be used to more completely mix first components 108a and second component 108b. The user may alternatingly press hollow second plunger 104 and plunger 154 in order to cycle the two-part composition back and forth between barrel 106 and second syringe barrel 152, resulting in more homogeneous mixing. Although shown with a second syringe 150 coupled to system 100, it is to be understood that sufficient mixing may be achieved simply within the syringe-in-syringe mixing system 100, particularly where the rupturable membrane or valve is configured to only pass first component 108a for mixing with second component 108b under a pressure sufficiently high to cause jetting of the first component into the second component (e.g., so as to create turbulence sufficient to mix the two components together). In such a configuration, the distal end of syringe barrel 106 may simply be capped during mixing within the syringe-in-syringe mixing system 100 (see FIG. 1). The cap 107 may include a check-valve or other vent (not shown) that permits air within barrel 106 to be expelled as first component 108a is expressed into barrel 106. Any check-valve known in the art can be used or modified to attach to barrel 106.

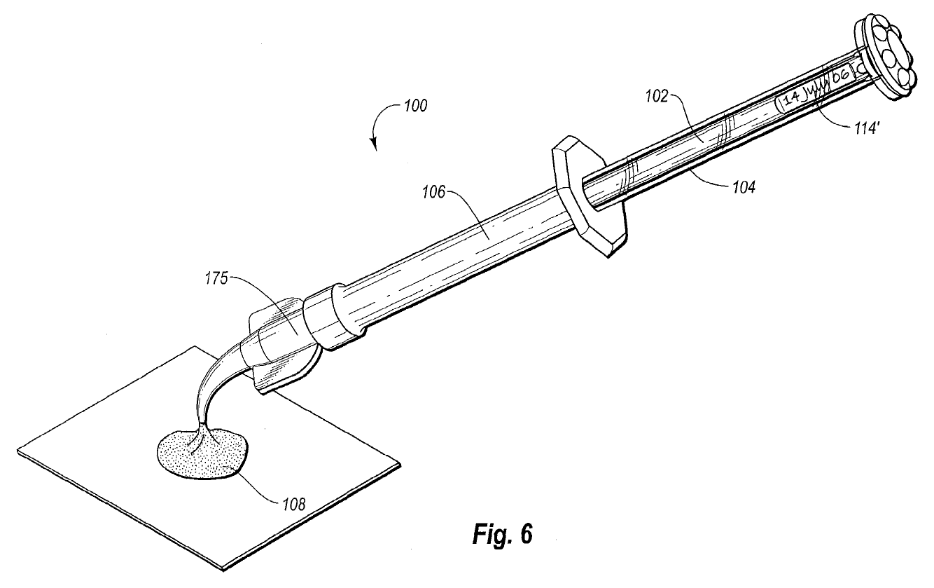

FIG. 6 illustrates the system 100 with a dispensing tip 175 coupled at a distal end of barrel 106 so as to allow the user to dispense the mixed two-part composition 108. In the case where a second syringe 150 is used to mix the components, the mixed composition is forced back into barrel 106, and second syringe 150 is detached from barrel before attaching dispensing tip 175. As illustrated, composition 108 maybe dispensed onto a pad for subsequent application (e.g., with a brush tool). Alternatively composition 108 may be dispensed directly onto a tooth or other surface, depending on the preference of the user.

FIG. 7A illustrates an alternative syringe-to-syringe mixing system 300 for mixing a first component 308a with a second component 308b. Such a system may be provided as a pre-dosed, prepackaged mixing solution in which the two components 308a and 308b are pre-measured in the correct quantities for mixing a desired quantity of the two-part composition. Pre-dosing and pre-packaging the components reduces the work required of the dental practitioner, and reduces the possibility that a user (particularly an inexperienced one) will make a mistake in measuring quantities of the components, which may drastically affect the effectiveness of the mixed composition. In addition, any such mistakes or variations may drastically alter the shelf-life of the mixed composition. In other words, although a composition may have a nominal shelf-life of about 30 days after mixing, because of a mistake in measurement of components, the composition may only have an actual shelf life of about 15 days, which will result in an ineffective composition if used after actual expiration but before the nominal expiration date.

System 300 is illustrated as similar to system 200 of FIG. 3D, but including a first plunger 302 similar to first plunger 202″ as illustrated in FIG. 3G. First plunger 302 is slidably disposed within first syringe barrel 304 containing a first component 308a, which is coupled to second syringe barrel 306 containing second component 308b. A second plunger 302a is slidably disposed within second syringe barrel 306. First and second chambers of first syringe barrel 304 and 306, respectively, are preferably separated by a valve, a rupturable barrier, or other separation means capable of initially keeping the two as components from mixing together prematurely. As seen in FIG. 7B, the user is able to record the mixing and/or expiration date 314a on label 314 prior to fully pressing first plunger 302 into first syringe barrel 304. As illustrated, the user may write a mixing and/or expiration date directly onto a label 314 (e.g., with a pen or marker). The label may include an adhesive (e.g., a pressure sensitive adhesive) so as to allow the user to adhere the label to the recessed portion 311 of stem 310, or alternatively, if the label includes no adhesive, the label 314 may be placed within recessed portion 311, and first plunger 302 inserted within first syringe barrel 204 so as to trap label 314 within the region of increased space defined between stem 310 and the wall of first syringe barrel 304. The region of increased space is sufficient to be capable of receiving a label on which a mixing and/or expiration date is recorded, and to permit sliding of the first plunger within the syringe barrel or hollow second plunger without scraping, rubbing, or otherwise removing the written date. For example, the region may measure at least about 0.0005 inch between an outside surface of the elongate stem and the interior surface of the wall defining the hollow first chamber, preferably between about 0.005 and about 0.15 inch, and more preferably between about 0.01 and about 0.08 inch.

In any case, the recorded date 314a is advantageously not located on a surface of stem 310 that will be rubbed against the inside surface of the wall of first syringe barrel 304, which may otherwise cause the date to be smeared, worn away, or otherwise rendered illegible. Rather than writing the date on a label, the user may write the date directly onto recessed surface 311. In addition, first syringe barrel 304 includes a transparent or translucent window portion (e.g., the whole syringe barrel 304 may be transparent or translucent) so that the practitioner can read the recorded date through the barrel 304 a (hollow second plunger.

Once the mixing and/or expiration date 314a has been recorded and label 314 has been positioned within recess 311, the user may press first plunger 302 into first syringe barrel 304 so as to compress first component 308a. As shown in FIG. 7C, once a sufficient force is applied, first component 308a is expressed under pressure from first syringe barrel 304 into second syringe barrel 306 where it mixes with second component 308b, which may occur through opening of a valve, rupture of a rupturable membrane, or breaking, opening, and/or parting of some other separation means, if present.

If necessary, the second plunger 302a may be used in conjunction with first plunger 302 to more completely mix first component 308a and second component 308b. The user may alternatingly press the first and second plungers in order to cycle the two-part composition back and forth between second syringe barrel 306 and first syringe barrel 304, resulting in more homogeneous mixing. Such additional mixing may not be necessary, particularly where any rupturable membrane, valve, or other separation means is configured to only pass first component 308a for mixing with second component 308b under a pressure sufficiently high to cause jetting of the first component into the second component (e.g., so as to create turbulence sufficient to mix the two components together). Once mixed, the composition may be dispensed for use as illustrated and described in conjunction with FIG. 6.

It will be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A time-indicating mixing, storing and dispensing system for use with a two-part dental composition that, when mixed together, yields a time-sensitive dental composition, the system comprising:
a first plunger comprising an elongate stem and a plug at a distal end of the elongate stem;
a hollow first chamber for containing a first component; the first chamber being defined by an interior surface of a wall, the hollow first chamber being adapted to receive the first plunger therein in slidable engagement
a hollow second chamber for containing a second component, the second chamber being adjacent to and initially separated from the first chamber such that first and second components are initially separate from each other;
means for providing a region of increased space between an outside surface of the elongate stem and the interior surface of the wall defining the hollow first chamber, the region of increased space being sufficient to receive a label on which is recorded a mixing date and/or an expiration date of a mixed two-part dental composition; and
means for protecting the recorded date from damage or alteration.

2. A time-indicating mixing, storing and dispensing system as recited in claim 1, wherein the means for providing a region of increased space comprises the elongate stem of the first plunger having a recessed portion such that a region of increased space exists between the elongate stem of the first plunger and the interior surface of the wall defining the hollow second chamber when the first plunger is received within the hollow second chamber such that a recorded date written on an outer surface of the elongate stem or written on a label inserted into the space is protected from damage or alteration once the first plunger is received within the hollow second chamber.

3. A time-indicating mixing, storing and dispensing system as recited in claim 2, wherein the recessed portion comprises an indented label surface formed into a portion of the elongate stem on which can be recorded a date.

4. A time-indicating mixing, storing and dispensing system as recited in claim 3, wherein at least a portion of the indented label surface comprises a thermoplastic elastomer material.

5. A time-indicating mixing, storing and dispensing system as recited in claim 4, wherein the thermoplastic elastomer material is overmolded over at least a portion of the first plunger.

6. A time-indicating mixing, storing and dispensing system as recited in claim 2, wherein the recessed portion extends around a circumference of the elongate stem such that the recessed portion has a cross-sectional diameter that is less than a cross-sectional diameter of an adjacent portion of the elongate stem.

7. A time-indicating mixing, storing and dispensing system as recited in claim 6, wherein the recessed portion is formed near a distal end of the first plunger.

8. A time-indicating mixing, storing and dispensing system as recited in claim 2, wherein the recessed portion extends around a circumference of the elongate stem such that the recessed portion has a cross-sectional diameter that is less than a cross-sectional diameter of an adjacent portion of the elongate stem, further comprising a hollow tube formed of a thermoplastic elastomer material that engages within the recessed portion having a reduced cross-sectional diameter, a region of increased space being defined between an outside surface of the hollow tube and the interior surface of the wall defining the hollow first chamber.

9. A time-indicating mixing, storing and dispensing system as recited in claim 1, further comprising a label on which the mixing and/or expiration date may be written by the user.

10. A time-indicating mixing, storing and dispensing system as recited in claim 9, wherein the label includes an adhesive for adhering the label to an outside surface of the first plunger.

11. A time-indicating mixing, storing and dispensing system as recited in claim 9, wherein the label comprises at least one of paper or a thermoplastic elastomer.

12. A time-indicating mixing, storing and dispensing system as recited in claim 1, wherein the hollow first chamber comprises a hollow second plunger that is adapted to receive the first plunger therein in slidable engagement and the hollow second chamber comprises a syringe barrel, the syringe barrel being adapted to receive the hollow second plunger therein in slidable engagement so as to allow syringe-in-syringe mixing of a first component with a second component.

13. A time-indicating mixing, storing and dispensing system as recited in claim 1, wherein the hollow first chamber comprises a first syringe barrel that is adapted to receive the first plunger therein in slidable engagement and the hollow second chamber comprises a second syringe barrel, the second syringe barrel being coupleable to the first syringe barrel so as to allow syringe-to-syringe mixing of a first component with a second component.

14. A time-indicating mixing, storing and dispensing system as recited in claim 1, wherein the means for providing a region of increased space comprises the hollow first chamber having a recessed portion of increased inside diameter such that a region of increased space exists between the elongate stem of the first plunger and the interior surface of the wall defining the hollow second chamber when the first plunger is received within the hollow second chamber such that a recorded date written on an outer surface of the elongate stem or written on a label inserted into the space is protected from damage or alteration once the first plunger is received within the hollow second chamber.

15. A time-indicating mixing, storing and dispensing system as recited in claim 1, further comprising separation means for initially separating a first component within the hollow first chamber from a second component within the hollow second chamber.

16. A time-indicating mixing, storing and dispensing system as recited in claim 15, wherein the separation means comprises a rupturable membrane.

17. A time-indicating mixing, storing and dispensing system as recited in claim 16, wherein the rupturable membrane comprises a thermoplastic elastomer.

18. A time-indicating mixing, storing and dispensing system as recited in claim 15, wherein the separation means comprises a valve.

19. A time-indicating mixing, storing and dispensing system as recited in claim 1, wherein the region of increased space measures at least about 0.0005 inch between an outside surface of the elongate stem and the interior surface of the wall defining the hollow first chamber.

20. A time-indicating mixing, storing and dispensing system as recited in claim 19, wherein the region of increased space measures between about 0.005 inch and about 0.15 inch.

21. A time-indicating mixing, storing and dispensing system as recited in claim 19, wherein the region of increased space measures between about 0.01 inch and about 0.08 inch.

22. A time-indicating mixing, storing and dispensing system for use with a two-part dental composition that, when mixed together, yield a time-sensitive dental composition, the system comprising:
a first plunger comprising an elongate stem and a sealing plug at a distal end of the elongate stem;
a hollow second plunger configured to contain therein a first component, the first plunger being slidably disposed within the hollow second plunger during use, the second plunger including a transparent or translucent window portion;
a syringe barrel configured to contain therein a second component, the hollow second plunger being slidably disposed within the syringe barrel during use; and
an indented label surface formed into a portion of the elongate stem on which can be recorded a mixing and/or an expiration date, an outer writing surface of the indented label surface comprising a thermoplastic elastomer,
wherein the mixing and/or expiration date recorded on the outer writing surface of the indented label surface can be viewed through the transparent or translucent window portion of the hollow second plunger once the first plunger is fully inserted into the hollow second plunger.

23. A method of mixing, storing and dispensing a time-sensitive composition formed by mixing together a two-part dental composition, the method comprising:
providing a time-indicating mixing, storing and dispensing system as recited in claim 1;
recording a mixing and/or expiration date on an outer surface of the elongate stem of the first plunger or on a label which is disposable within the region of increased space between the outside surface of the elongate stem and the interior surface of the wall of the hollow first chamber; and
pressing the elongate stem of the first plunger into the hollow first chamber so as to cause the first component within the hollow first chamber to be mixed with the second component within the hollow second chamber.

24. A method as recited in claim 23, wherein the mixing and/or expiration date is written directly onto the outer surface of the elongate stem of the first plunger.

25. A method as recited in claim 23, wherein the mixing and/or expiration date is written on an adhesive label, the method further comprising adhering the adhesive label to an outside surface of the elongate stem.

26. A method as recited in claim 23, wherein the mixing and/or expiration date is written on a label without any adhesive, the method further comprising inserting the label into the region of increased space between the elongate stem of the first plunger and the interior surface of the wall defining the hollow first chamber such that the label is free-floating and confined within the region of increased space, the step of inserting the label being performed prior to fully pressing the elongate stem of the first plunger into the hollow first chamber so as to cause the first component within the hollow first chamber to be mixed with the second component within the hollow second chamber.
